(19) United States
(12) Patent Application Publication
(10) Pub. No.: US 2007/0107087 A1
(43) Pub. Date: May 10, 2007

(54) Use of early light-inducible proteins (elip) to increase plant resistance to photochemical oxidant stress

(75) Inventors: Laurent Nussaume, La Tour d'Aigues (FR); Michel Havaux, Ansouis (FR); Claire Hutin, Paris (FR)

Correspondence Address: LOWE HAUPTMAN BERNER, LLP, 1700 DIAGONAL ROAD, SUITE 300, ALEXANDRIA, VA, 22314, US

(73) Assignee: Comissariat A L'Energie Atomique, Paris (FR)

(21) Appl. No.: 10/505,757

(22) PCT Filed: Mar. 3, 2003
(86) PCT No.: PCT/FR03/00673

§ 371 Date: Feb. 2, 2005

(30) Foreign Application Priority Data
Mar. 1, 2002 (FR) 0202623

(51) Int. Cl. A01H 5/00; A01H 1/00; G01N 33/53

(52) U.S. Cl. 800/289; 435/7.1; 530/370; 800/320

(57) ABSTRACT

A method for increasing resistance of a plant to photooxidative stress, which entails the steps of transforming the plant with at least one polynucleotide encoding a protein of the ELIP family, and expressing the protein in the plant.

The present invention relates to novel means for increasing plant resistance to photooxidative stress.

The exposure of plants to a high light intensity can lead to a light absorption which exceeds the capacities of use by the photosynthetic function, resulting in the appearance of active oxygen species (superoxide, hydrogen peroxide, hydroxyl). These molecules are very reactive and, if they are not detoxified, they cause oxidative stress which modifies the structure and the function of the chloroplast, resulting in an inhibition of photosynthetic activity. This phenomenon can manifest itself even under moderate light intensities when the plant is exposed to low temperatures (WISE, Photosynth. Res. 45, 79-97, 1995), or else when it is placed under conditions of drought (SMIRNOFF, New Phytol., 125, 27-58, 1993), or of mineral deficiency.

Photooxidative stress is harmful to plants, for example to tropical varieties which are relatively unsuited to the cold, or else to plants which are planted during the first days of spring and must therefore grow at low temperature. The observed symptoms are : leaf bleaching, appearance of necroses, reduction in growth.

Thus, the search for novel means for combating this photooxidative stress constitutes a real need.

Biochemical analyses of plants after exposure to a high intensity light show an accumulation of various proteins belonging to the family of chlorophyll a/b binding LHCPs (ADAMSKA et al., Eur. J. Biochem., 260, 453-460, 1999). Among these proteins, mention will be made of those of the family of ELIPs (Early Light Induced Proteins) (ADAMSKA, Physiologia Plantarum, 100, 794-805, 1997).

For example, 2 ELIP proteins, respectively called ELIP1 and ELIP2, have been demonstrated Arabidopsis thaliana (At). Sequences homologous to those of these ELIPs have also been identified in other plants; in the case of cereals, mention will in particular be made of rice (Os), maize (Zm) and wheat (Wh).

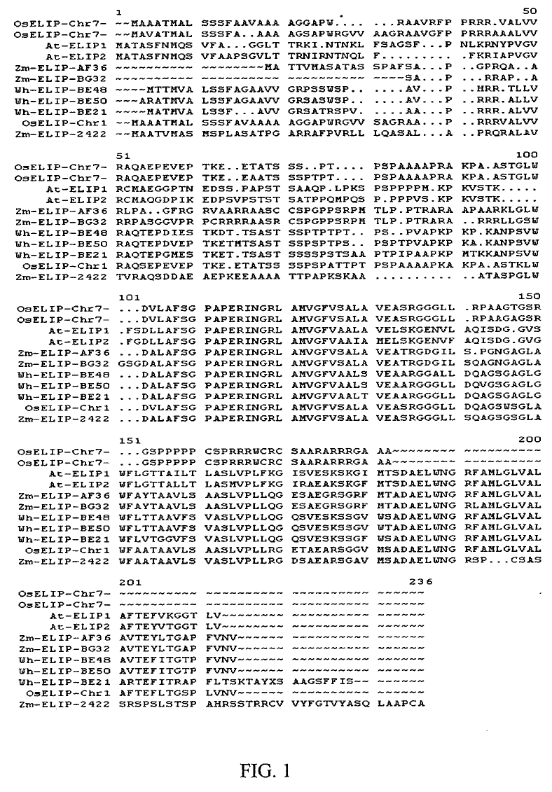

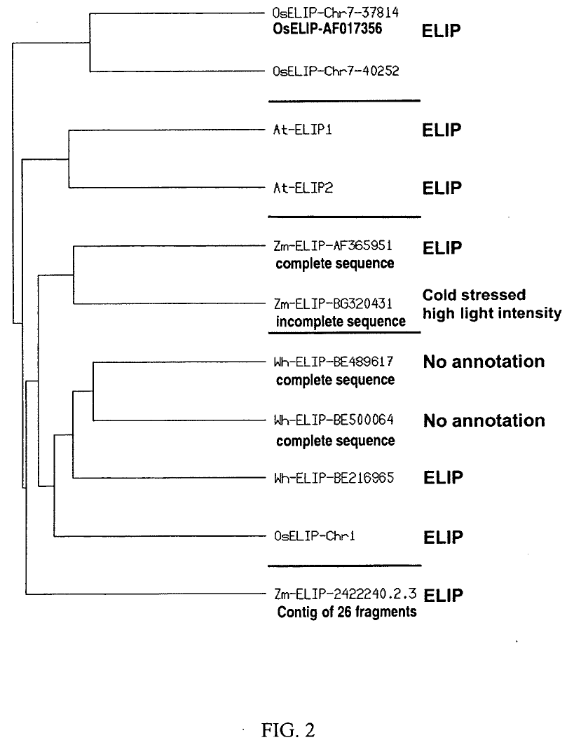

FIG. 1 represents the sequence alignment of some ELIPs. The genealogical tree for these ELIPs, developed by means of sequences representative of the various classes, is represented in FIG. 2.

The rice ELIPs can be divided up into 2 classes, encoded respectively by genes located on chromosome 1 and chromosome 7 (2 genes have been identified on chromosome 7). The maize ELIPs can also be divided up into 2 classes; on the other hand, the wheat ELIP sequences currently available appear to belong to one and the same class. Some classes comprise several ESTs or genes, exhibiting slight variations, implying that there is a polymorphism for these genes.

ELIPs have a, transient expression ; their genes are only mainly transcribed in response to various environmental stresses (strong light intensity, low temperature, specific stages of development of the plant, ABA) (ADAMSKA, 1997, publication mentioned above).

The amount of ELIPs in plant cells is also tightly controlled by post-translational regulation : integration of ELIPs into the thylakoid membrane, instability and degradation of ELIPs (ADAMSKA, 1997, publication mentioned above).

Various observations suggest a correlation between stress and the presence of ELIPs. On the other hand, it had never, up until now, been possible to determine whether these proteins played an effective role in resistance to stress. On the contrary, it had been reported that a decrease in or a partial suppression of expression of ELIPs does not result in any particular phenotype (GRUBER, Doctoral Thesis: <<Untersuchung der expression eines fruhen lichtinduzierten Proteins des Tabaks >>, University of Hanover, 1999, cited in MONTANE et al., Gene, 258, 1-8, 2000).

Some authors have, in addition, focused on the pathway for transporting ELIPs to the thylakoid membranes : a spontaneous insertion mode has been proposed by KRUSE et al. (Eur. J. Biochem., 208, 195-202, 1992), and KIM et al. (J. Biol. Chem., 274, 4715-4721, 1999), who adds thereto the involvement of a pathway involving nucleotide triphosphates. However, these hypotheses, based on in vitro experiments, have not been confirmed in vivo.

In the present invention, the inventors have demonstrated a preferential pathway for the transport and integration, in vivo, of ELIPs into the thylakoid membranes of chloroplasts ; this pathway involves a transporter complex called cpSRP (for. Chloroplast Signal Recognition Particle).

The inventors have thus observed that mutations which inactivate the cpSRP43 subunit (chaos mutant) of the cpSRP complex affect all ELIPs. In plants containing the chaos mutation, the ELIPs no longer accumulate under conditions of photooxidative stress, and a substantial increase in sensitivity to the stress is observed in parallel.

In addition, the inventors have also noted that the overexpression of ELIPs in these mutants restores a resistance to photooxidative stress which is comparable to that of the wild-type plants.

These results make it possible to propose the use of ELIPs in applications where resistance to photooxidative stress is desired, in particular in the plant field.

A subject of the present invention is thus the use of at least one protein of the ELIP family, or of a polynucleotide encoding said protein, for obtaining plants having increased resistance to photooxidative stress.

In particular, a subject of the present invention is a method for increasing the resistance of a plant to photooxidative stress, comprising the transformation of said plant with at least one polynucleotide encoding a protein of the ELIP family, and the expression of said protein in said plant.

This method can be implemented by the usual methods, known in themselves, of genetic engineeering and plant transgenesis.

Conventionally, use will be made of an expression cassette in which will be placed a polynucleotide encoding the ELIP protein intended to be expressed, under the control of appropriate sequences for regulating the expression (in particular transcription promoter and terminator). The gene of interest may also be combined with other regulatory elements, such as activators. Other elements such as introns, enhancers, polyadenylation sequences and derivatives may also be present in the nucleic acid sequence of interest, in order to improve the expression or the functioning of the transforming gene. The expression cassette may also contain 5′ untranslated sequences referred to as <<leader >> sequences. Such sequences can improve translation.

The CsVMV (cassava vein mosaic virus) promoter described in PCT application WO 97/48819 can, for example, be used.

A heterologous promoter can also be used; a very large number of promoters which can be used for transforming plant cells are known in themselves. By way of nonlimiting examples of promoters which can be used in the context of the present invention, mention will be made:
of strong constitutive promoters, such as the cauliflower mosaic virus (CaMV) 35S promoter described by KAY et al. (Science, 236, 4805, 1987), or its derivatives, the rice actin promoter followed by the rice actin intron (RAP-RAI) contained in the plasmid pActl-T (McELROY et al., Mol. Gen. Genet., 213, 150-160, 1991), or the ubiquitin promoter;
of inducible promoters, for example light-inducible promoters, such as that of the small subunit of ribulose bisphosphate carboxylase of various plant species, cold-inducible promoters, or drought-inducible promoters, such as that of ASR (ABA-water stress-ripening-induced protein) described in PCT application WO 01/83756.

Among the transcription terminators which can be used, mention will in particular be made of the 35S polyA terminator of CaMV (FRANCK et al., Cell, 21, 285-294, 1980; Gene Bank n° V 00141), or the NOS polyA terminator, which corresponds to the 3′ noncoding region of the nopaline synthase gene of the Agrobacterium tumefaciens nopaline strain Ti plasmid (DEPICKER et al., J. Mol. Appl. Genet., 1, 561-573, 1982).

The expression cassette thus obtained can optionally be inserted into a vector capable of replicating in a host cell, or of inserting into the chromosomal DNA thereof.

According to one embodiment, the expression cassette is inserted into a nucleotide vector, such as a plasmid, which can also comprise a marker gene, for example a gene for selecting a transformed plant from a plant which does not contain the transfected foreign DNA.

Among the genes encoding a selection agent (also called selector marker genes), use may in particular be made of genes which confer resistance to an antibiotic (Herrera-Estrella et al., 1983) such as hygromycin, kanamycin, bleomycin or streptomycin, or to herbicides (EP 242 246) such as glufosinate, glyphosate or bromoxynil.

Preferably according to the invention, the promoter combined with the gene encoding a selection agent is a constitutive promoter, such as the actin promoter-actin intron, corresponding to the 5′ noncoding region of the rice actin 1 gene and its first intron (Mc Elroy et al., 1991; Gene Bank n° S 44221). The presence of the first actin intron makes it possible to increase the level of expression of a gene when it is fused 3′ of a promoter. This promoter sequence allows, for example, constitutive expression of the NptII gene.

Preferably, said gene encoding a selection agent is excized. The excision system for eliminating the gene encoding a selection agent can be a transposition system, such as in particular the maize Ac/Ds system, or recombination system, such as in particular the P1 bacteriophage Cre/lox system, the yeast FLP/FRT system (Lyzrik et al., 1997), the Mu phage Gin recombinase, the E. coli Pin recombinase or the pSR1 plasmid R/RS system. A cotransformation system (Komari et al., 1996) can also be used. Preferably, the system used will be the maize Ac/Ds system.

According to a preferred embodiment, said gene is included within the Ds transposable element. The Ds transposon used is described in the publication by Yoder et al., (1993). The Ds element is an Ac element which has undergone considerable mutations or deletions in the sequence encoding the transposase. It can excize itself from its insertion site only in the presence of an active transposase source Ac. It is therefore Ac-dependent. A preferred system for eliminating a selection marker gene can comprise two components:
a first plant having no active transposase, into which a construct comprising the cassette for expressing the gene of interest and for expressing the gene encoding a selection agent, bordered by the mobilizable sequences of a transposon, can be integrated;
a second plant containing in its genome a gene encoding an endogenous active transposase.

The crossing of these two plants results in the obtaining of regenerants, obtained from F1 plants or from F2 plants selected for the presence of the gene of interest without selection marker gene.

The transformation of plant cells can be carried out by various methods, such as, for example, transfer of the polynucleotide of interest into plant protoplasts after incubation of the latter in a solution of polyethylene glycol in the presence of divalent cations (Ca 2+), electroporation (FROMM et al., Proc. Nat. Acad. Sci., 82, 5824, 1985), the use of a particle gun, in particular according to the method described by FINER et al. (Plant Cell Report, 11, 323-328, 1992), or cytoplasmic or nuclear microinjection (NEUHAUS et al., Theor. Appl. Genet., 75(1), 30-36, 1987).

It is also possible to infect the plant cells with a bacterial cellular host comprising the vector containing the polynucleotide of interest. The cellular host may be Agrobacterium tumefaciens (AN et al., Plant Physiol., 81, 86-91, 1986), or A. rhizogenes (GUERCHE et al., Mol. Gen. Genet., 206, 382, 1987). In this case, preferably, the transformation of the plant cells is carried out by transferring the T region of the A. tumefaciens tumor-inducing extrachromosomal circular plasmid Ti, using a binary system (WATSON et al., recombinant DNA, Ed. De Boeck University, 273-292, 1994).

For the transformation of monocotyledons, the method described by ISHIDA et al. (Nature Biotechnology, 14, 745-750, 1996) may also, for example, be applied.

The resistance to photooxidative stress of the plants transformed according to the invention, compared to control plants, can be assessed using various morphological, physiological and/or biochemical methods of measurement, for specific stress conditions.

The invention also relates to the transgenic plants obtained by means of a method in accordance with the invention, and also to the descendants of these plants. The invention also encompasses the products obtained from these plants, such as plant tissues or organs, cells, seeds, etc.

The hybrid transgenic plants., obtained by crossing at least one plant according to the invention with another, are also part of the invention.

The present invention can also be used in the context of the selection of plants having improved resistance to photooxidative stress.

They are of most particular value in the context of marker-assisted selection (MAS), which makes it possible to use accelerated backcross techniques consisting in using the linkage which exists between a molecular marker and a gene of agronomic interest, in this case encoding an ELIP, for transferring said gene of interest into various genotypes in order to provide them with increased resistance to photooxidative stress.

The present invention can also be used to monitor the integration of an allele of the ELIP gene that is favorable to resistance to photooxidative stress, for example in the context of introgression techniques by means of successive crosses between plants having this allele.

The present invention can also be used in the context of phylogenetic studies, of the characterization of genetic relationships among plant varieties, of the identification of somatic hybridizations or crosses, of the localization of chromosomal segments affecting monogenic characteristics, of cloning based on genetic maps, and of the identification of QTLs (Quantitative Trait Loci) and/or of PQL (Protein Quantitative Loci).

A subject of the present invention is thus the use of at least one protein of the ELIP family, or of an antibody directed against said protein, or of a polynucleotide representing all or part of the gene of said protein, for evalulating the resistance of a plant to photooxidative stress.

In this context, a subject of the invention is in particular a method for evaluating the resistance of a plant to photooxidative stress, characterized in that said plant is subjected to a photooxidative stress, and in that the level of expression, by said plant, of at least one protein of the ELIP family is determined.

In order to subject a plant to a photooxidative stress, it is placed under environmental conditions which promote the production of active oxygen species by means of light. These conditions may, for example, be obtained by subjecting the plant to a very strong light, optionally combined with cold, and/or to drought, and/or to a mineral deficiency.

The level of expression of the proteins of the ELIP family can be evaluated, for example, by quantifying the transcripts of one or more ELIP genes; this evaluation can be readily carried out by means of various techniques known in themselves, for example using oligonucleotides derived from the sequences of these genes.

It can also be evaluated by directly assaying the ELIP protein(s) concerned, using for example conventional immunoassay techniques, of the type ELISA, immunoelectrophoretic transfer, etc.

Anti-ELIP antibodies which can be used for implementing this method may be readily obtained by means of conventional methods for preparing polyclonal or monoclonal antibodies, by immunizing an animal with an ELIP protein extracted from plants, or else produced by genetic engineering.

It is also possible to identify ELIP gene alleles that are favorable to resistance to photooxidative stress, using plant lines already known to naturally exhibit a high resistance to this type of stress. These alleles can be isolated and sequenced and their sequences can be compared with those of the ELIP genes originating from less tolerant varieties, in order to identify polymorphisms associated with these alleles, for example polymorphisms located in the coding sequence or in the promoter of the gene. Nucleotide probes and/or primers for detecting these polymorphisms can then be used for selecting plants having these favorable alleles.

The methods in accordance with the invention for identifying plants having increased resistance to photooxidative stress can be implemented in particular in the context of marker-assisted selection, or for following the introgression into plants of a characteristic of resistance to photooxidative stress.

The present invention can be used in all plant species in which an increase in resistance to photooxidative stress is desirable. They may be dicotyledons or monocotyledons, in particular cereals, ornamental plants, or plants intended for human or animal food, etc. It is of most particular advantage in the case of plants of tropical or subtropical origin (for example maize, cucumber and tomato) which are very sensitive to damage caused by photooxidative stress when they are grown in cold climates.

The present invention will be understood more thoroughly from the further description which follows, which refers to nonlimiting examples showing the ability of ELIP proteins to increase plant resistance to photooxidative stress.

### EXAMPLE 1

### Transport of ELIPs to Thylakoid Membranes VIA The CPSRP Pathway

The inventors studied ELIP transport in a double mutant deficient for the cpSRP43 and 54 subunits of the cpSRP pathway.

I- Obtaining the chaos/ffc double mutant

1) Crossing of 2 Single Mutants Chaos and ffc

Two Arabidopsis thaliana mutants are used
the chaos single mutant deficient for the cpSRP43 subunit of the cpSRP transport complex by virtue of the insertion of a Ds element at the 3′ end of the coding region of the chaos gene (KLIMYUK et al., Plant Cell, 11, 87-99, 1999);
the ffc single mutant deficient for the cpSRP54 subunit of the cpSRP transport complex by virtue of the insertion of a 10 kb DNA fragment into intron 8 of the ffcl-2 gene (AMIN et al., Plant Physiol., 121, 61-71, 1999).

A cross between the chaos and ffc single mutants produces a wild-type F1 generation.

Double mutants (chaos/ffc) deficient for the cpSRP54 and 43 subunits are selected from the F2 generation.

2) Selection of the Chaos/ffc Double Mutants

The membrane proteins of wild-type (WT), single mutant (ffc, chaos) and double mutant (chaos/ffc) Arabidopsis thaliana are extracted as described by GILLET et al. (Plant. J., 16, 257-262, 1998) and the concentration of said proteins is assayed using the Lowry method (Sigma-Aldrich).

10 μg of each of the membrane protein extracts are separated by polyacrylamide gel electrophoresis under denaturing conditions and are transferred onto nitrocellulose membranes as described by AMIN et al., 1999, mentioned above.

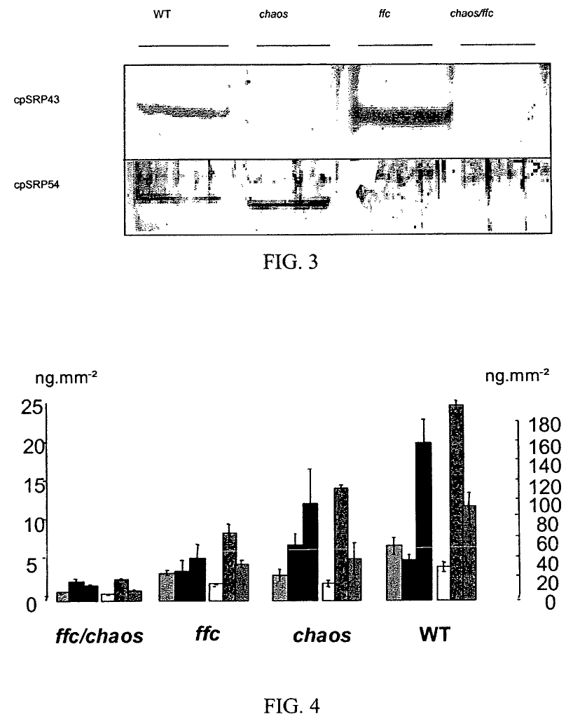

The membranes are incubated with anti-cpSRP43 (dilution at 1:10 000) and anti-cpSRP54 (dilution at 1:2 500) antibodies coupled to alkaline phosphatase, and then visualized by means of the alkaline phosphatase ligand. The results are represented in FIG. 3.

Legend of FIG. 3

line cpSRP43=detection with anti-cpSRP43 antibody
line cpSRP54=detection with anti-cpSRP54 antibody
lane WT=wild-type
lane chaos=chaos single mutant (cpSRP43⁻)
lane ffc=ffc single mutant (cpSRP54⁻)
lane chaos/ffc=chaos/ffc double mutant (cpSRP43⁻/cpSRP54⁻).

The results reveal a band corresponding to the presence of the cpSRP43 subunit in the WT and the ffc single mutant, and a band corresponding to the presence of cpSRP54 subunit in the WT and chaos single mutant.

The results show that the effects of the mutations in the ffc and chaos single mutants are additives in the ffc/chaos double mutant. The chaos/ffc double mutant is deficient for the cpSRP 43 and 54 subunits (cpSRP43⁻/cpSRP54⁻double mutation).

II- Characterization of the ffc/Chaos Double Mutant

After in vi tro germination of the seeds at 23/17° C. (day/night), for a photoperiod of 8 hours/day, the 2-week-old young plants are transferred into soil, in a growth chamber at 23/17° C. (day/night, under a photon flux density of 300 μmol/m²/second, with a photoperiod of 8 hours/day and 60/85% humidity.

All the analyses are carried out on leaves collected at the vegetative stage, before flowering (rosette stage).

1) Phenotypic Analysis

The chaos and ffc single mutants exhibit pale green chlorosis due to the loss of photosynthetic pigments.

The chaos/ffc double mutants contain much less pigment than one or other of the single mutants and, as a result of this, appear to be more yellow than the latter.

2) Physiological Analyses

a) Analysis of the Photosynthetic Pigments

Disks 0.8 cm in diameter are cut out of the leaves derived from wild-type (WT), single mutant (chaos, ffc) and double mutant (chaos/ffc) Arabidopsis plants, and then frozen in liquid nitrogen.

The photosynthetic pigments are analyzed by HPLC according to the method described by HAVAUX and TARDY (Plant Physiol., 113, 913-923, 1996). The results are represented in FIG. 4.

Legend of FIG. 4

along the x-axis:
ffc/chaos=cpSRP54⁻/cpSRP43⁻double mutant
ffc=cpSRP54⁻single mutant
chaos=cpSRP43⁻single mutant
WT=wild-type
along the y-axis: photosynthetic pigments (ng/mm²)
[symbol] =chlorophyll a
[symbol] =chlorophyll b
[symbol] =neoxanthine
▪=violaxanthine, antheric xanthine, zeaxanthine
□=lutein
[symbol] =β-carotene

The results show that the chaos and ffc single mutants have lost 40% and 60%, respectively of their photosynthetic pigments. The chaos/ffc double mutant shows an 85% decrease in chlorophylls, a 67% decrease in carotenoids and an 87% decrease in β-carotene. However, the decrease in these pigments does not alter the chlorophyll a/b ratio, which is similar in the chaos/ffc double mutants and the WT control (2.0±0.1).

b) Analysis of the Ultrastructure of the Chloroplasts and of the Thylakoids

Leaf samples of approximately 1 mm² are fixed as described by CARDE et al. (Biol. Cell, 44, 315-324, 1982). The micrographs of these samples are produced with a CM10 or TECNAI (FEI) transmission electron microscope at 80 kV.

The electron micrographs of the chloroplasts of the chaos/ffc double mutants reveal a considerable decrease in the size of the grana and of the lamellae compared to the WT, unlike the chloroplast ultrastructure in the ffc and chaos single mutants, which is not altered. However, the size and the number of the chloroplasts per cell, determined by confocal microscopy, are not different from the WT. As a result of this, the decrease in pigmentation in the double mutant is associated with a deficient development of the thylakoid membranes.

c) Analysis of the Chlorophyll Concentration in Photosynthetic Systems I and II (PSI and PSII)

The fluorescence emission spectra for the chlorophyll of thylakoid membrane suspensions are recorded in liquid nitrogen (77 K).

The thylakoid membranes are prepared as described by ROBINSON and YOKUM (Biochem. Biophys. Acta, 590, 97-106, 1980), and the chlorophyll concentration of the membrane preparations is determined in 80% acetone (ARNON, Plant Physiol., 24, 1-15, 1949).

The chlorophyll fluorescence emission spectra are measured by means of a fiber-optic spectrofluorimeter (Perkin-Elmer LS50B) using the thylakoid preparations diluted to chlorophyll concentrations of 50 μg/ml in 20 mM HEPES/NaOH, 3 mM MgCl₂, pH 7.5. The fluorescence emission spectra for chlorophyll generally have 3 distinct peaks : peaks at 680 nm and 690 nm due to the chlorophyll a associated with PSII (PSII internal chlorophyll a antenna apoproteins CP43 and CP47 with a contribution from the LHCII proteins), and a peak at 730 nm due to the chlorophyll a associated mainly with PSI (LHCI proteins) (GOVINDJEE, Aust. J. Plant Physiol., 22, 131-160, 1995).

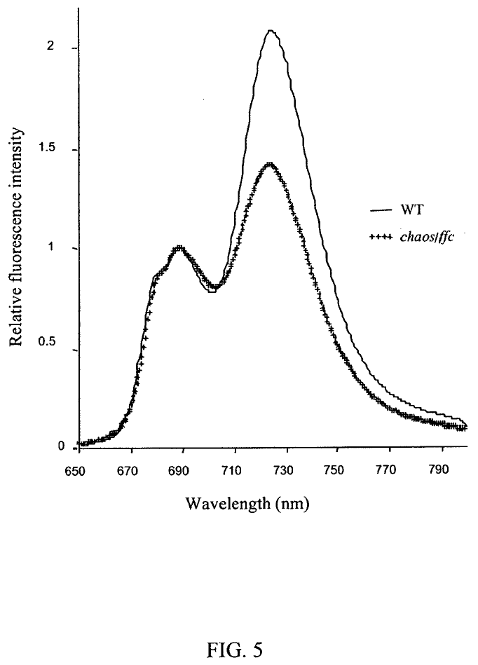

The thylakoid membranes (400 μl) are frozen in liquid nitrogen and the chlorophyll fluorescence emission is excited at 440 nm. The results are represented in FIG. 5.

Legend to FIG. 5

along the x-axis: wavelength (nm)
along the y-axis: relative fluorescence intensity
_{———}=wild-type (WT)
+++=cpSRP43⁻/cpSRP54⁻(chaos/ffc) double mutant

The results show that the chlorophyll fluorescence emission spectra exhibit 3 distinct peaks at approximately 680 nm (F680), 690 nm (F690) and 730 nm (F730). The F680 and F690 peaks are attributed to the chlorophylls associated with the PSII photosystem. The F730 peak is attributed to the chlorophyll molecules associated mainly with the PSI photosynthetic system. The results also show that the PSI fluorescence peak (F730) is significantly reduced in the chaos/ffc double mutant compared to the WT, unlike the PSII fluorescence bands (peaks F680 and F690), suggesting a drastic decrease in the LHCI proteins, by comparison with PSII.

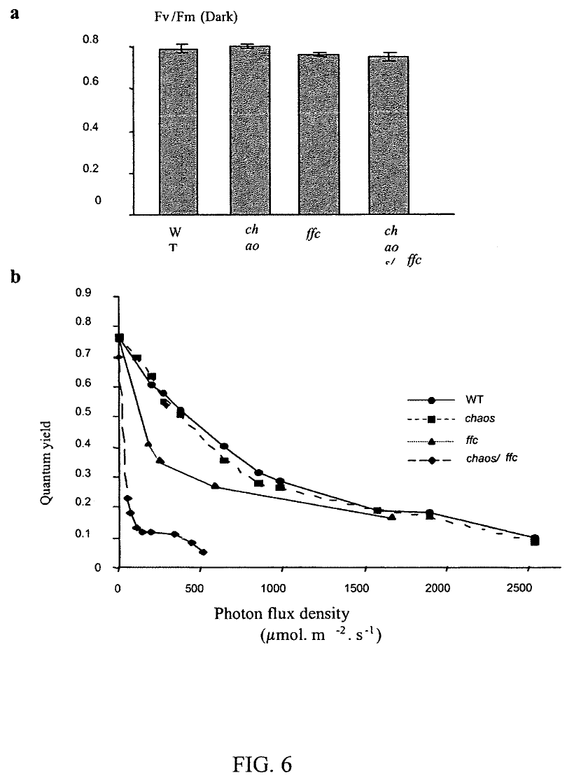

The maximum photochemical efficiency of PSII is determined in vivo by measuring the fluorescence of the chlorophyll (Fv/Fm) in dark-adapted leaves for 30 minutes, at ambient temperature, as previously described by KLIMYUK et al., 1999, mentioned above. The results are represented in FIG. 6a.

Legend of FIG. 6a

along the x-axis :
WT=wild-type
chaos=cpSRP43⁻single mutant
ffc=cpSRP54⁻single mutant
chaos/ffc=cpSRP43⁻/cpSRP54⁻double mutant
along the y-axis photochemical efficiency: Fv/Fm (dark)

These results show that the fluorescence parameter Fv/Fm of the double mutant (chaos/ffc) is similar to that measured in the leaves of the wild-type (WT) and of the single mutants (chaos, ffc), indicating that the PSII photosystem is correctly assembled and photochemically competent in the double mutant.

The real quantum yield of the PSII photochemistry (Φ) in leaves exposed to light. is evaluated in the presence of increasing photon flux densities. The results are represented in FIG. 6b,.

Legend to FIG. 6b

along the x-axis: photon flux density (μmol/m²/sec)
along the y-axis: quantum yield
●=wild-type (WT)
▪=cpSRP43⁻(chaos) single mutant
▴=cpSRP54⁻(ffc) single mutant
♦=cpSRP43⁻/cpSRP54⁻(chaos/ffc) double mutant.

The results show that the quantum yield of the PSII photochemistry of the double mutant is very different from the other genotypes (WT, single mutants), indicating that a photosynthetic transport of electrons in the leaves of the double mutant become rapidly saturated as the photon flux density increases, reaching a quantum yield of PSII of virtually zero at a density of approximately 500 μmol/m²/sec. The electron transport is not inhibited in the chaos single mutant compared with the WT (similar quantum yields), whereas the ffc single mutant exhibits an intermediate electron transport between that of the WT and that of the chaos single mutant.

All these results indicate, in the double mutant, blocking of electron transport at a site different from the PSII system, suggesting that this transport is limited by the low amount of PSI complexes.

3) Biochemical Analyses

a) Analysis of the Amount of LHCPs and of ELIPs

The analysis of the LHCP and ELIP contents is carried out in plants at the rosette stage, under normal conditions (CC: photon flux density of 300 μmol/m²/sec) or subjected to a light stress (LL: photon flux density of 5 μmol/m²/sec ; HL: photon density of 500-1000 μmol/m²/sec, respectively).

LHCPs

The membrane proteins are extracted and analyzed by Western blotting as described in section I-2). The membrane protein samples are loaded onto the gel at 2 or 3 different concentrations in order to ensure linearity of the calorimetric immunodetection. Each experiment is repeated at least 3 times.

In a first series of experiments, the amount of LHCPs is evaluated in the plants under normal conditions (CC).

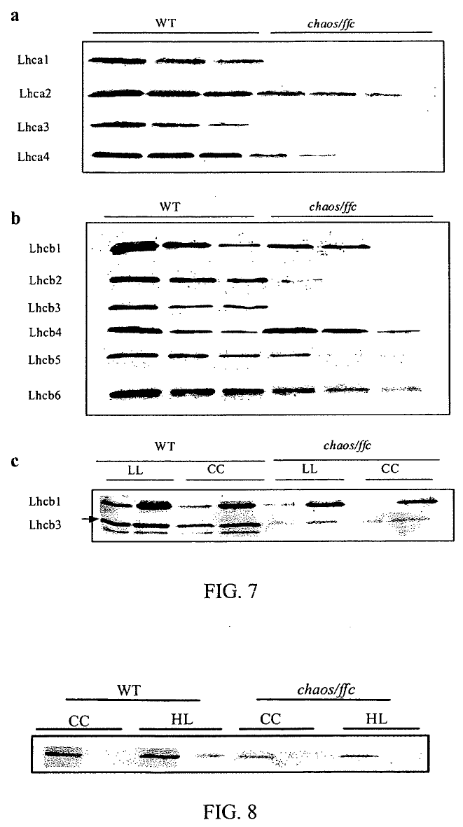

The dilution of the polyclonal sera used is indicated in brackets followed by the amounts of membrane protein loaded onto the gel: anti-Lhcal (1:3000) 5/3.5/2.5 μg; anti-Lhca2 (1:200) 5/3.5/2.5 μg; anti-Lhca3 (1:1000) 2.5/1.5/0.75 μg; anti-Lhca4 (1:3000) 10/7/5 μg; anti-Lhcb1 (1:5000) 1/0.5/0.2 μg; anti-Lhcb2 (1:1000) 1.5/1/0.75 μg; anti-Lhcb3 (1:30) 3/2/1.5 mg anti-Lhcb4 (1:2500) 2.5/1/0.5 μg; anti-Lhcb5 (1:100) 1.5/1/0.75 mg; anti-Lhcb6 (1:750) 6/4/3 μg. The results are represented in FIGS. 7a and 7b.

Legend of FIGS. 7a and 7b

lanes 1 to 3=wild-type (WT)
lanes 4 to 6=double mutant (chaos/ffc)
(a) line Lhca1=detection with anti-Lhcal antibodies
line Lhca2=detection with anti-Lhca2 antibodies
line Lhca3=detection with anti-Lhca3 antibodies
line Lhca4=detection with anti-Lhca4 antibodies
(b) line Lhcb1=detection with anti-Lhcb1 antibodies
line Lhcb2=detection with anti-Lhcb2 antibodies
line Lhcb3=detection with anti-Lhcb3 antibodies
line Lhcb4=detection with anti-Lhcb4 antibodies
line Lhcb5=detection with anti-Lhcb5 antibodies
line Lhcb6=detection with anti-Lhcb6 antibodies.

The results show that, under normal conditions, all the LHCP contents tested are decreased by the cpSRP43⁻/cpSRP54⁻double mutation, with the exception of the Lhcb4 protein, the content of which represents approximately 130% of that of the WT. The Lhcal, Lhca3 and Lhcb3 proteins are no longer detected in the chaos/ffc double mutant. The Lhca4, Lhcb2 and Lhcb5 proteins are detected in trace amounts, not exceeding 10% of the content of the WT. The Lhca2, Lhcb1 and Lhcb6 proteins are detected in an amount equivalent to 50% of the content of the WT.

In a second series of experiments, the content of Lhcb1 and Lhcb3 proteins was evaluated in the plants under normal conditions (CC) and under conditions of light stress (LL).

The dilution of the sera used is indicated (between brackets) followed by the amounts of membrane proteins loaded into the gel : anti-Lhcb1 (1 :5000) 1/0.2 μg; anti-Lhcb3 (1 :30) 3/1.5 μg. The results are represented in FIG. 7c.

Legend of FIG. 7c

lanes 1 to 4=wild-type (WT)
lanes 5 to 8=double mutant (chaos/ffc)
lanes 1, 2, 5 and 6=growth under conditions of light stress (LL)
lanes 3, 4, 7 and 8=growth under normal conditions (CC)
line Lhcb1=detection with anti-Lhcb1 antibodies
line Lhcb3=detection with anti-Lhcb3 antibodies.

The results show that the WT exhibits a slight decrease in the LHCP content when the light intensity decreases. On the other hand, the double mutant exhibits Lhcb3 protein contents that are undetectable whatever the light intensity, and the Lhcb1 protein content, although slightly increased when the light intensity is low, always remains less than that of the WT at the same light intensity.

The results show that the decrease in the LHCP content is due to the absence of the cpSRP43 and 54 subunits and not due to a secondary effect generated in the double mutant by light stress.

ELIPs

The ELIP content was evaluated in the plants under normal conditions (CC) and under conditions of light stress (HL).

The extraction and the quantification of the proteins were carried out as described by PÖTTER and KLOPPSTECH, Eur. J. Biochem., 214, 779-786, 1993.

Detection of the ELIPs at two concentrations (20 and 0.5 μg) is carried out using an anti-ELIP rabbit serum diluted to 1:2000 and a goat anti-rabbit secondary antibody coupled to alkaline phosphatase (Sigma, Munich, Germany). The results are represented in FIG. 8.

Legend of FIG. 8

lanes 1 to 4=wild-type (WT)
lanes 5 to 8=double mutant (chaos/ffc)
lanes 1, 2, 5 and 6=growth under normal conditions (CC)
lanes 3, 4, 7 and 8=growth under conditions of light stress (HL)

The results show that very few ELIPs are detected in the chaos/ffc double mutant compared with the WT, whatever the light intensity.

All these results indicate that the cpSRP43⁻/cpSRP54⁻double mutation affects the LHCP and ELIP contents in a similar manner, and that the cpSRP pathway is involved in transport of the LHCPs and of the ELIPs to the thylakoid membranes.

b) Analysis of the Amount of Proteins Imported Via Pathways Other Than cpSRP

Besides the cpSRP pathway, 3 other pathways for transporting proteins from the stroma to the thylakoid membranes or the lumen exist: the ΔpH and Sec pathways and the spontaneous insertion pathway.

The influence of the cpSRP43⁻/cpSRP54⁻double mutation is evaluated by means of detection using antibodies specific for proteins imported specifically via each of these pathways : the OE23 protein transported by the ΔpH pathway, the psbS and psbW proteins transported by the spontaneous insertion pathway and the PC protein transported by the Sec pathway. The influence of the double mutation is also evaluated with respect to the chloroplast envelope protein (E37) and a component of the chloroplast transport machinery (ClpC).

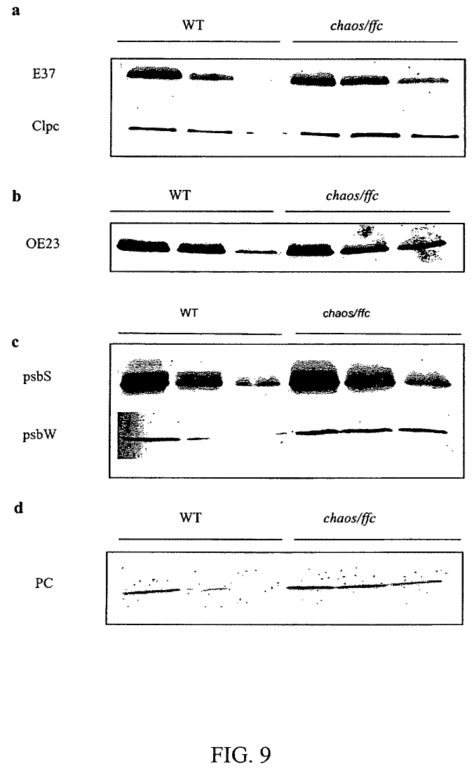

The dilution of the polyclonal sera used is indicated (between brackets) followed by the amounts of membrane protein loaded onto the gel: anti-E37 (1:6000) 10/5/2 μg, anti-ClpC (1:4000) 5/3.5/2 μg, anti-PC (1:5000) 10/5/2 μg, anti-OE23 (1:10 000) 3/1.25/0.5 μg, anti-psbS (1:4000) 10/5/2 μg, anti-psbW (1:2000) 60/40/30 μg. The results are represented in FIGS. 9a, 9b, 9c and 9d.

Legend of FIG. 9a

line E37=detection with anti-E37 antibodies
line Clpc=detection with anti-Clpc antibodies
lanes 1 to 3=wild-type (WT)
lanes 4 to 6=cpSRP43⁻/cpSRP54⁻(chaos/ffc) double mutant.

The results show a 30% excess of the E37 and Clpc proteins in the chaos/ffc double mutant compared to the WT.

Legends of FIGS. 9b, 9c and 9d

lanes 1 to 3=wild-type (WT)
lanes 4 to 6=cpSRP43⁻/cpSRP54⁻(chaos/ffc) double mutant
(a) line OE23=detection with anti-OE23 antibodies
(b) line psbS=detection with anti-psbS antibodies
line psbW=detection with anti psbW antibodies
(c) line PC=detection with anti-PC antibodies.

The results show, for the 3 transport pathways ΔpH, Sec and spontaneous insertion pathway, higher amounts of proteins imported specifically by each of these pathways in the double mutant, compared with the WT.

These results show that the pathways for transport to the thylakoids or the lumen, other than the cpSRP pathway, are not affected by the double mutation.

III- Conclusion

The ELIPS are not present in the double mutant. They are also absent in the cpSRP43 single mutant. On the other hand, they are present in the cpSRP54 single mutant, at a level comparable to the WT (normal level).

According to the results of the above analyses, the cpSRP43 subunit is involved in the transport of ELIPs to the thylakoid membranes.

In addition, the study of the amounts of LHCPs and of ELIPs, by physiological and biochemical analyses, shows the independent but additive role of the subunits of the cpSRP complex.

These results show that the importation of the ELIPs into the thylakoid membranes occurs predominantly, or even exclusively, via the cpSRP pathway.

Biochemical analyses: The amount of LHCPs decreases by half in the single mutants, and is virtually zero in the double mutants.

The amount of ELIPs is virtually zero in the double mutant, and also in the cpSRP43⁻ single mutant.

The proteins conveyed via the other 3 pathways (spontaneous insertion, ΔpH and Sec) are present in amounts comparable to the WT in the double and single mutants.

Microscopic analyses: The number and the shape of the chloroplasts are the same in the mutants and the WT. The thylakoid structure is affected only in the double mutant. No notable difference is observed between the cpSRP43⁻ single mutant and the WT.

Photosynthesis is not inhibited in the cpSRP 43⁻ single mutant, unlike that of the cpSRP43⁻/cpSRP54⁻ double mutant.

A very small amount of ELIPs is found in the double mutant, indicating that the cpSRP pathway is involved in the transport of these proteins to the thylakoid membranes. The chaos single mutant, which is affected for the LHCPs, also integrates fewer ELIPs. Thus, the double mutation affects the LHCPs and the ELIPs.

### EXAMPLE 2

### ROLE of the ELIPs in Resistance to Photooxidative Stress

I-Choice of a Study Model: the cpSRP43 Mutant

1) The Chaos (cpSRP43⁻) Mutant

The chaos single mutant was identified in a population consisting of 217 independent lines of Arabidopsis thaliana ecotype Landsberg erecta containing an element derived from the maize dissociation disposable element (Ds). Self-pollinization of a plant of the 348/74/A line results in a lineage exhibiting a recessive chlorotic mutant phenotype (KLIMYUK et al., Pol. Gen. Genet., 249, 357-365, 1995). The mutant was called chaos. A pale coloration is observed throughout the vegetative cycle of the plant and uniformly affects all the tissues above ground.

2) Analysis of the ELIP Content Under Conditions of Photooxidative Stress

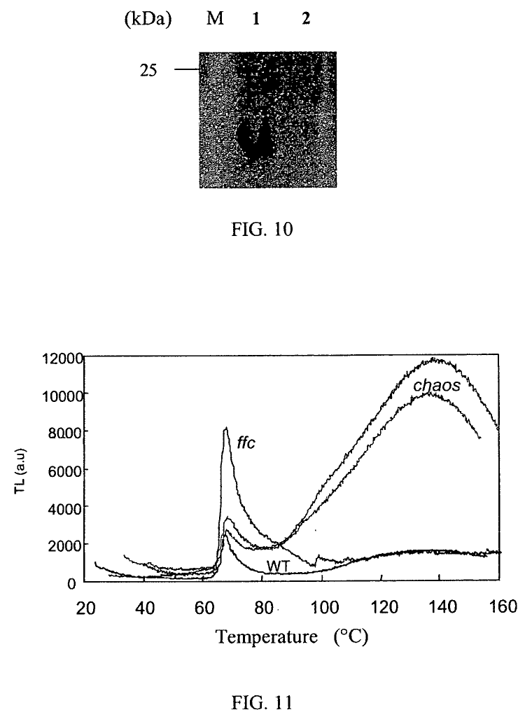

4- to 5-week-old plants, carrying the cpSRP43⁻ single mutation (chaos mutant), are subjected to a light stress (1000 μmol/m²/sec) combined with a cold stress (6-7° C.) for 4 days, with a photoperiod of 8 hours/day. The membrane proteins are extracted and 10 μg are analyzed by Western blotting as described in section I-2) of Example 1. The results are represented in FIG. 10.

Legend of FIG. 10

lane M=molecular weight marker (kDa)
lane 1=membrane proteins of the WT
lane 2=membrane proteins of the chaos mutant

The results show that, during the stress, the wild-type control accumulates ELIPs, unlike the chaos mutant, which is almost totally devoid of ELIPs.

On the other hand, the ELIPs are always present in the cpSRP54⁻ mutants (result not shown).

3) Observations of the cpSRP43⁻(Chaos Mutant) and cpSRP54⁻ (ffc Mutant) Lines Under Conditions of Photooxidative Stress

The analyses of the lines are carried out in plants at the rosette stage, under normal conditions (50 μmol/m²/sec at 23° C.) or subjected to a light stress (1000 μmol/m²/sec) combined with a cold stress at 6-7° C.

a) Phenotypic Analysis of the Chaos and ffc Mutants

Under normal conditions, the chaos and ffc mutants are pale green in color compared with the WT.

Under stress conditions, the appearance of anthocyanin pigments is noted in the WT and the ffc mutant.

On the other hand, the appearance of necroses and of white marks is noted in the chaos mutant, indicating a sensitivity to the oxidative stress.

b) Biochemical Analysis of the Chaos (cpSRP43-) and ffc (cpSRP54-) Mutants

The phenotypic results are confirmed by means of experiments to measure the thermoluminescence of the chlorophyll using a Hamamatsu photomultiplier tube (R36) as described by HAVAUX, Israel J. Chem., 38, 246-256, 1998.

The samples of leaves dark-adapted for 15 minutes are heated from 25 to 170° C., at a rate of 7° C./minute. The results are represented in FIG. 11.

Legend of FIG. 11

along the x-axis: temperature (° C.)
along the y-axis: thermoluminescence (au=arbitrary units)
W=wild-type
chaos=cpSRP43⁻ single mutant (2 allelic mutants)
ffc=cpSRP54⁻ single mutant

The results show that the thermoluminescence of the ffc single mutant is similar to that of the WT. On the other hand, the thermoluminescence of 2 chaos single allelic mutants is considerably increased compared to the WT.

The results indicate that the ffc mutant is resistant to the photooxidative stress in the same way as the WT, on the other hand, the chaos mutant is sensitive to the photooxidative stress.

4) Control of the Nature of the Stress

The same experiments were carried out in the absence of oxygen.

The results show a disappearance of the symptoms described above in section 3) of Example 2. This control confirms that it is indeed an oxidative stress which is the cause of such symptoms. These results therefore suggest a correlation between the sensitivity to the oxidative stress and the absence of ELIPs.

II-overexpression of ELIPs in the cpSRP43⁻ Mutant

To demonstrate the relationship between the presence of ELIPs and the resistance to the photooxidative stress, specific ELIP complementation of the line lacking the cpSRP 43 subunit (chaos mutant) was carried out. This experiment was carried out to a successful conclusion due to the overproduction of ELIPs by chaos mutant (cpSRP 43⁻).

1) Vectors and Preparation of the Constructs Used

The sequences encoding two ELIPs, ELIP1 and ELIP2, respectively, are amplified from two genomic clones of Arabidopsis thaliana (GenBank accession numbers AB022223 and Z97336) by RT-PCR using the following primers:

Primers for ELIP1

(SEQ ID NO: 12)

| | |
|---|---|
| in the 3′ | CGGGATCCTTTAGCTTTAGACTAGAGTCCC |
| position | |

(SEQ ID NO: 13)

| | |
|---|---|
| in the 5′ | ATAAGAATGCGGCCGCCATGCAGTCAGTTTTCGCTGCT |
| position | |
| Primers for ELIP2 | |

(SEQ ID NO: 14)

| | |
|---|---|
| in the 3′ | CGGGATCCATTCATGGGCAAATCGTATTAA |
| position | |

(SEQ ID NO: 15)

| | |
|---|---|
| in the 5′ | ATAAGAATGCGGCCGCAATGGCAACAGCATCGTTCAAC |
| position | |

The sequences underlined result in the insertion of the BamHI restriction site (G/GATCC) in the 3′ position, and of the NotI restriction site (GC/GGCCGC) in the 5′ position of the PCR fragments.

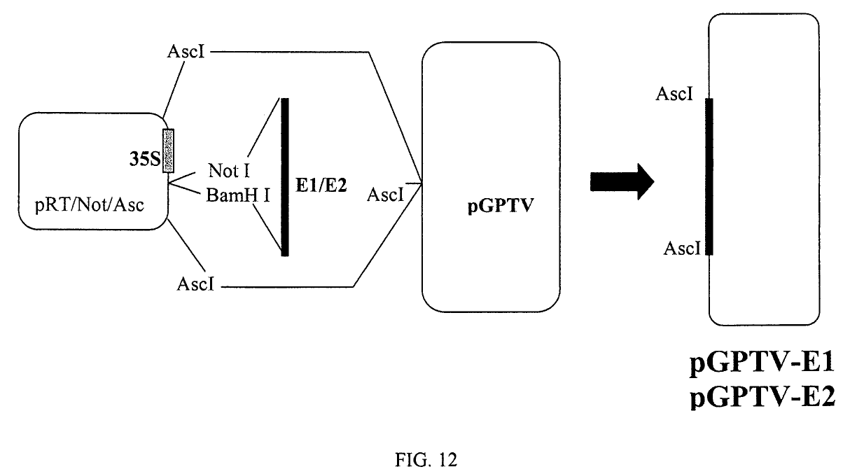

The main cloning steps are shown diagrammatically in FIG. 12.

Each of the PCR fragments is integrated into the vector pRT-Ω/NotI/AscI, which derives from the vector pRT 100 as described by OBERLACKER and WERR (Molecular Breeding, 2, 293-295, 1996), downstream of the 35S promoter: P35S˜ELIP1 (E1) and P35S˜ELIP2 (E2). Once the chimeric construct has been created in pRT-Ω/NotI/AscI, it is combined with a selection marker (hygromycin resistance gene). The E1 and E2 fragments flanked by AscI restriction sites are ligated to the unique SmaI site, converted beforehand to an AscI site, of the vector PGPTV, and thus allow transfer of the entire expression cassette into the binary vector (pGPTV-E1 and pGPTV-E2).

2) Transformation of Arabidopsis

The transformation of cpSRP43-deficient Arabidopsis thaliana (chaos mutant) with the pGPTV-E1 or pGPTV-E2 is carried out via Agrobacterium tumefaciens, by infiltration under vacuum (CLOUGH and BENT, The Plant Journal, 16, 735-743, 1998). Most of the descendants (chaos transformants) are genetically uniform (non-chimeric) and the clonal variation associated with the culturing of tissues and the regeneration thereof is minimized. The transformed lineage is hemizygous for the transgene at a given locus.

3) Biochemical Analysis of the ELIP Content of Chaos Transformants

The level of expression of the ELIPs in the absence of stress in chaos transformants containing the 35S-ELIP2 construct was estimated by Western blotting.

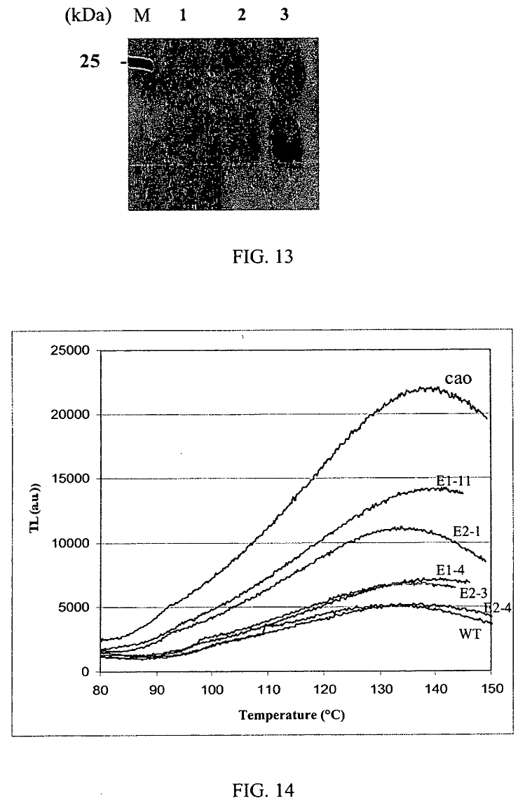

The results are represented in FIG. 13.

Legend of FIG. 13

lane M=molecular weight marker (kDa)
lane 1=chaos mutant
lanes 2 and 3=chaos transformants E2-3 and E2-4, respectively; the band corresponding to the ELIPs is indicated with an arrow.

The results show that the 2 transformants are capable of accumulating the ELIPs, unlike the chaos mutant. They also indicate that the E2-4 transformant (lane 3) accumulates more ELIPs than the E2-3 transformant (lane 2). Although the main pathway needs chaos proteins, overexpression with a strong promoter results in an accumulation of ELIPs.

4) Observations of the Chaos Transformants

a) Phenotypic Analysis

4- to 5-week-old plants of the chaos mutant and of the two chaos transformants E2-3 and E2-4 are subjected to a light stress (1000 μmol/m²/sec) combined with a cold stress (6-7° C.) for 4 days, with a photoperiod of 8 hours/day.

Under conditions of cold/light stress, a range of resistance which correlates with the level of expression of the ELIPs can be noted.

This range is characterized by necroses and white marks in the sensitive plants (chaos mutants), which decrease in the transgenic plants which have acquired intermediate resistance (E2-3 transformant) and disappears in the E2-4 transformant, which reflects the restoration of the resistance to the photooxidative stress.

These results indicate that the chaos transformants containing the 35S-ELIP2 construct are capable of developing resistance to the cold/light stress, and that this resistance depends on the level of expression of the transgene.

b) Biochemical Analysis

The analysis of the thermoluminescence of the chlorophyll in chaos transformants containing the 35S-ELIP2 construct is carried out as described in section I-3) b) of Example 2.

The results are represented in FIG. 14.

Legend of FIG. 14

along the x-axis: temperature (° C.)
along the y-axis: thermoluminescence (au=arbitrary units)

wt=wilde-type

cao=chaos mutant

E1-11, E2-1, E1-4, E2-3, E2-4=transformants at various levels of expression of ELIPs (E1-ll<E2-l<E1-4<E2-3<E2-4).

The results show that the thermoluminescence of the chlorophyll is at a maximum (>2000) for the chaos mutant sensitive to the photooxidative stress and at a minimum (<5000) for the WT resistance to photooxidative stress. The chaos transformants El-11, E2-1, El-4 and E2-3 exhibit a variable thermoluminescence of between 1400 and 6000, indicating partial resistance to the photooxidative stress. Only the E2-4 transformant exhibits a thermoluminescence equal to that of the WT, indicating a reappearance of a WT-type resistance to the photooxidative stress.

These results confirm that expression of the ELIPs confers resistance to the photooxidative stress.

5) Model Proposed

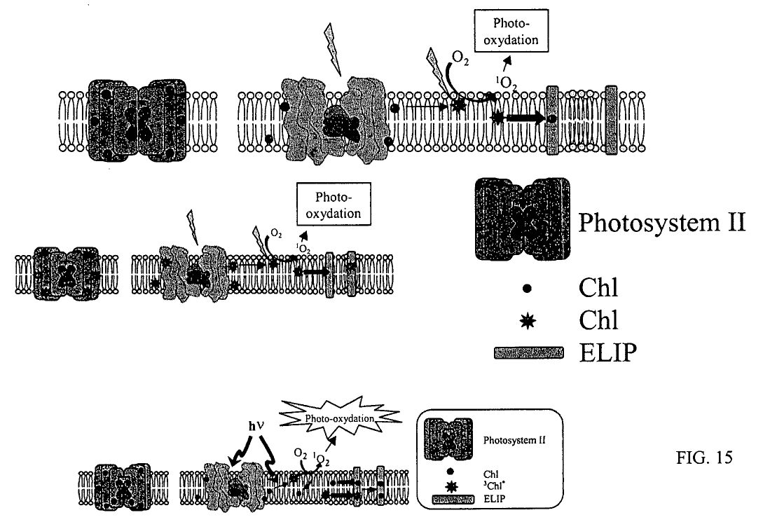

These results are in accordance with one of the hypotheses for the function of ELIPs, which are thought to trap free chlorophylls a, thus preventing the production of active oxygen species, and consequently providing protection against photooxidation. This model is represented in FIG. 15.

6) Activity of the Photosystems

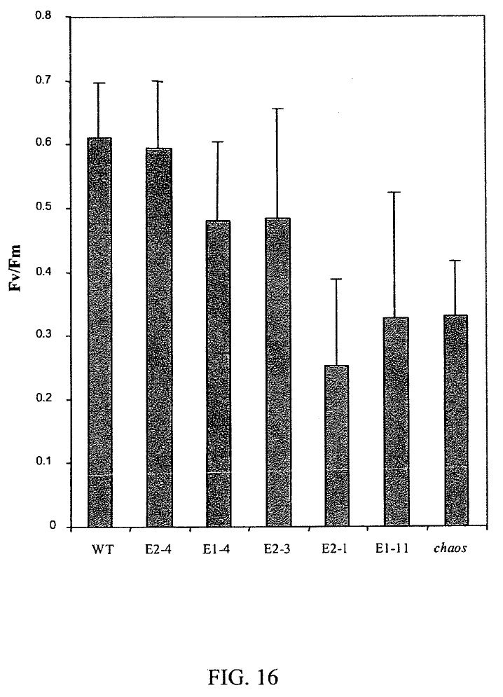

The activity of photosystem II (PSII) is evaluated by measuring the fluorescence of the chlorophyll (Fv/Fm) as described in section II- 2) c) of Example 1. The results are represented in FIG. 16.

Legend of FIG. 16

along the x-axis : wild-type (wt), chaos mutant (chaos), chaos transformants (El-11, E2-1, E2-3, E1-4 and E2-4) along the y-axis : maximum quantum yield of the photochemistry of PSII measured by means of the fluorescence parameter (Fv/Fm)

The results show the photochemical efficiency of PSII for the WT. The E1-11 and E2-1 transformants have a PSII photochemical activity similar to that of the chaos mutant (approximately 0.3); the E1-4 and E2-3 transformants have an intermediate PSII photochemical activity (approximately 0.5); only the E2-4 transformant has a PSII photochemical activity comparable to that of the WT (approximately 0.6).

These results indicate that the presence of ELIPs preserves the photosystems.

### EXAMPLE 3

### Transformation of Maize—Overexpression of ELIPs in Maize

In order to confer better resistance to photooxidative and/or cold stress on maize plants, the Arabidopsis genes encoding the ELIPs (ELIP 1 and/or ELIP 2), under the control of a strong constitutive promoter, are introduced into and expressed ectotopically in transformed and regenerated maize.

I- Vectors and Preparation of the Constructs Used

The sequences encoding two ELIPs, ELIP1 and ELIP2, are amplified from two genomic clones of Arabidopsis thaliana (GenBank accession numbers AB022223 and Z97336) respectively by RT-PCR using the following primers:

Primers for ELIP1:

(SEQ ID NO: 12)

| | |
|---|---|
| in the 3′ | CGGGATCCTTTAGCTTTAGACTAGAGTCCC |
| position | |

(SEQ ID NO: 16)

| | |
|---|---|
| in the 5′ | ATAAGAATGAATTCATGCAGTCAGTTTTCGCTGCT |
| position | |

Primers for ELIP2:

(SEQ ID NO: 14)

| | |
|---|---|
| in the 3′ | CGGGATCCATTCATGGGCAAATCGTATTAA |
| position | |

(SEQ ID NO: 17)

| | |
|---|---|
| in the 5′ | ATAAGAATGAATTCAATGGCAACAGCATCGTTCAAC |
| position | |

The sequences underlined result in the insertion of the BamHI restriction site (G/GATCC) in the 3′ position and of the EcoRI restriction site (G/AATTC) in the 5′ position, of the PCR fragments.

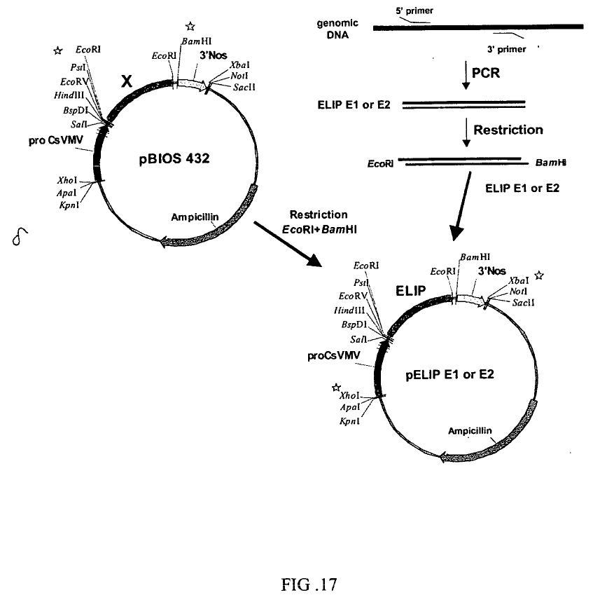

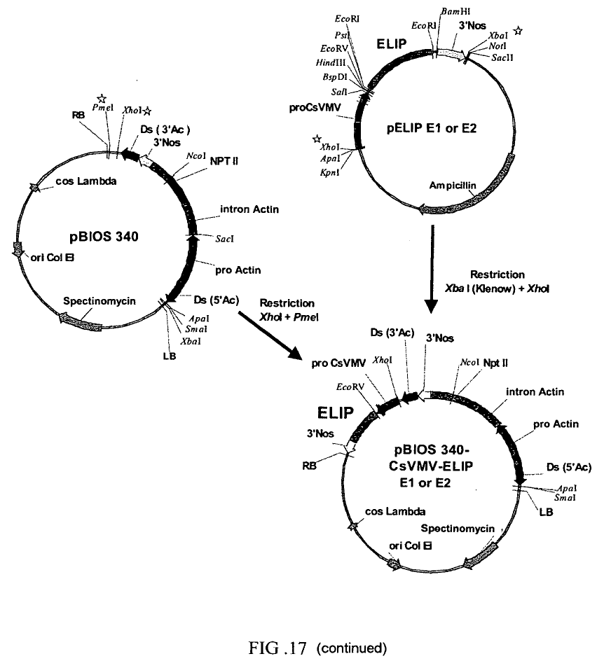

The main cloning steps are shown diagrammatically in FIG. 17.

Each of the PCR fragments is integrated downstream of the CsVMV promoter between the EcoRI and BamHI sites of the vector pBIOS 432, and upstream of the NOS polyA terminator, in a pBluescriptII-type vector from Stratagene. This involves replacing the existing gene between the promoter and the terminator of the plasmid pBIOS 432 with the ELIP-E1 or ELIP-E2 gene. Once these chimeric constructs have been made, they are combined with a selection marker (kanamycin resistance gene). For this, the CsVMV promoter—ELIP E1 or E2—polyA NOS terminator cassettes are isolated as Xbal (site blunt-ended with klenow) and XhoI fragments and introduced into the vector pBIOS 340 digested with the Pme I (blunt ended) and Xho I enzymes, and then dephosphorylated with SAP. The vector pBIOS 340 is a vector containing the plasmid sequences of the vector pSB12 (Japan Tobacco, EP 672 752) and the cassette Ds element—actin promoter—actin intron—NPTII gene—Nos terminator—DS element (PCT application WO 02/101061). The vectors pBIOS 340-CsVMV-ELIP-E1 and pBIOS 340-CsVMV-ELIP-E2 are thus generated and will be used to generate the <<super-binary >> vector used to transform the maize with Agrobacterium.

The vector used to transform the maize with Agrobacterium tumefaciens is in the form of a superbinary plasmid of approximately 50 kb.

The superbinary vector used for the transformation contains:
an ori region: plasmid origin of replication Col EI, required for the maintenance and for the multiplication of the plasmid in Escherichia coli. This origin of replication is not functional in Agrobacterium tumefaciens,
an origin of replication that is functional in Agrobacterium tumefaciens and in Eschericia coli,
the cos region of the lambda bacteriophage, which may be of use for manipulating the vector in vitro,
the additional virB, virC and virG regions of Agrobacterium tumefaciens, which increase the efficiency of transformation,
the genes for resistance to tetracyclin (Tetra) and to spectinomycin (Spect), which are expressed only in the bacteria,
a T-DNA carrying ELIP E1 or E2 genes, and the NptII gene inserted into a Ds element, conferring kanamycin resistance, these two genes being functionally linked to elements for their transcription. In the present example, the ELIP E1 or E2 gene is under the control of the CsVMV promoter and of the Nos polyA terminator; the NptII gene inserted into a Ds element being under the control of the actin promoter and of the first intron and of the Nos polyA terminator.

The NptII gene was isolated from the Tn5 transposon of Escherichia coli (BERG et al., Genetics, 105, 813-828, 1983). This gene encodes the enzyme neomycin phosphotransferase type II which catalyzes the O-phosphorylation of aminoglycoside antibiotics such as neomycin, kanamycin, gentamycin and G418 (DAVIES and SMITH, Annu. Rev. Microbiol., 32, 469-518, 1978). This gene confers resistance to kanamycin, which is used as a selection agent in plant genetic transformation. It is described by Bevan et al. (Genbank n°U00004).

This superbinary vector is obtained by homologous recombination of an acceptor plasmid pSBl (EP 672 752) derived from an Agrobacterium tumefaciens Ti plasmid, with the donor plasmids pBIOS 340-CsVMV-ELIP-E1 or pBIOS 340-CsVMV-ELIP-E2 derived from pUC (MESSING, Methods Enzymol., 101, 20-78, 1983).

II- Transformation of Maize

The maize is transformed according to the protocol described by ISHIDA et al. (Nature Biotechnology, 14, 745-750, 1996), in particular using immature embryos taken 10 days after fertilization. All the media used are referenced in the cited reference. The transformation begins with a coculturing phase in which the immature embryos of the maize plants are brought into contact for at least 5 minutes with Agrobacterium tumefaciens LBA 4404 containing the superbinary vectors. The superbinary plasmid is the result of a homologous recombination between an intermediate vector carrying the T-DNA containing the gene of interest and/or the selection marker derived from the plasmids described in the previous examples, and the vector pSB1 from Japan Tobacco (EP 672 752) which contains : the virB and virG genes of the plasmid pTiBo542 present in the supervirulent Agrobacterium tumefaciens strain A281(ATCC 37349) and a homologous region found in the intermediate vector which allows this homologous recombination. The embryos are then placed on LSA medium for 3 days in the dark and at 25° C. A first selection is carried out on the transformed calluses the embryogenic calluses are: transferred onto LSD5 medium containing phosphinotricin at 5 mg/l and cefotaxime at 250 mg/l (elimination or limitation of the contamination with Agrobacterium tumefaciens). This step is carried out for 2 weeks in the dark and at 25° C. The second selection step is carried out by transferring embryos which have developed on LSD5 medium, onto LSD10 medium (phosphinotricin at 10 mg/l) in the presence of cefotaxime, for 3 weeks under the same conditions as previously. The third selection step consists in excizing the type I calluses (fragments of 1 to 2 mm) and in transferring them onto LSD 10 medium in the presence of cefotaxime, for 3 weeks in the dark and at 25° C.

The plantlets are regenerated by excizing the type I calluses which have proliferated and transferring them onto LSZ medium in the presence of phosphinotricin at 5 mg/l and cefotaxime for 2 weeks at 22° C. and under continuous light.

The plantlets which have regenerated are transferred onto RM+G2 medium containing 100 mg/l of Augmentin® (amoxicillin/clavulanic acid) for 2 weeks at 22° C. and under continuous light for the development step. The plants obtained are then transferred to a phytotron for the purpose of acclimatizing them.

The transformed linear is hemizygous for the transgene at a given locus.

In order to identify the plantlets which are resistant to kanamycin and have therefore integrated the transgene, a selection step (cornet drop test) is carried out with a solution of kanamycin at a concentration of 500 mg/l, to which 1% Tween has been added. 2 to 3 drops of this solution are applied to the maize plants at the 4 to 5-leaf stage.

The plants are analyzed 5 days after application of the kanamycin. The sensitive plants exhibit the appearance of whitish zones (death of the chlorophyll-containing tissues). The resistant plants do not exhibit the appearance of whitish zones 5 days after application of the kanamycin.

Molecular Characterization of the Transformants

Southern methodology (1975) is used to demonstrate the insertion of the transgene into the genome of the plant and to evaluate the number of copies and characterize the integration profile.

The genomic DNA is extracted from the leaves of the plants according to a CTAB extraction protocol, according to the protocol of Keller J (DNAP 6701 San Pablo Ave Oakland Calif. 94608 USA) modified by Bancroft I. (Department of Molecular, Genetics, Cambridge Laboratory, John Innes Center for Plant Science Research, Colney lane, Norwich, England). The DNA obtained is digested with the NcoI restriction enzyme, separated by agarose gel electrophoresis and transferred onto a hybond N+membrane and then hybridized with radioactive probes (CsVMV probe and actin-intron probe). The transformants are thus characterized.

The plantlets which are resistant to kanamycin and have therefore integrated the transgene exhibit increased resistance to photooxidative stress.

The gene encoding the selection agent (NptII gene) is advantageously eliminated according to the Ac/Ds elimination system (PCT application WO 02/101061).

1-10. (canceled)

11. A method for increasing resistance of a plant to photooxidative stress, which comprises the steps of transforming said plant with at least one polynucleotide encoding a protein of the ELIP family, and expressing said protein in said plant.

12. The method of claim 11, wherein said at least one polynucleotide encoding a protein of the ELIP family is in an expression cassette.

13. The method of claim 12, wherein the expression cassette comprises leader sequences.

14. The method of claim 11, wherein said at least one polynucleotide encoding a protein of the ELIP family is selected from the group consisting of ELIP-E1 and ELIP-E2.

15. The method of claim 12, wherein said expression cassette comprises CsVMV promoter.

16. The method of claim 11, wherein said plant is of tropical or sub-tropical origin.

17. The method of claim 16, wherein said plant is a cereal.

18. The method of claim 17, wherein said cereal is maize.

19. The method of claim 17, wherein said plant is rice.

20. The method of claim 17, wherein said plant is wheat.

21. The method of claim 16, wherein said plant is a tomato plant.

22. The method of claim 11, wherein said at least one polynucleotide encoding a protein of the ELIP family is integrated into thylakoid membranes of chloroplasts.

23. A transgenic plant which is obtained by the method of claim 11.

24. The transgenic plant of claim 23, which is a plant of tropical or sub-tropical origin.

25. The transgenic plant of claim 24, which is a cereal.

26. The transgenic plant of claim 25, which is maize.

27. The transgenic plant of claim 25, which is rice.

28. The transgenic plant of claim 25, which is wheat.

29. The transgenic plant of claim 25, which is a tomato plant.

30. A product obtained from the transgenic plant of claim 23, which product is selected from the group consisting of plant tissues or organs, cells and seeds.

31. A method for evaluating resistance of a plant to photooxidative stress, which comprises the steps of subjecting the plant to a photooxidative stress, and determining a level of expression, by said plant, of at least one protein of the ELIP family.

32. The method of claim 31, wherein the level of expression of said protein of the ELIP family is determined by immunoassaying the protein.

33. A hybrid transgenic plant produced by crossing at least one plant of claim 23 with another plant.

34. A method of selecting a plant for resistance to photooxidative stress, which comprises the steps of detecting at least one protein of the ELIP family in plants, and selecting one or more plants based upon said detection.

35. The method of claim 34, wherein said detecting comprises detecting antibodies against said at least one protein of the ELIP family.

36. The method of claim 34, wherein said detecting comprises detecting at least one polynucleotide encoding all or part of a gene for said at least one protein of the ELIP family.


SEQUENCE LISTING (17 sequences)

SEQ ID NO: 1 - protein, 156 aa, Oryza sativa
MAAATMALSSSFAAVAAAAGGAPWRAAVRFPPRRRVALVVRAQAEPEVEPTKEETATSSSPTPSPAAAAPRAKPAASTGLWDVLAFSGPAPERINGRLAMVGFVSALAVEASRGGGLLRPAAGTGSRGSPPPPPCSPRRRWCRCSAARARRRGAAA

SEQ ID NO: 2 - protein, 164 aa, Oryza sativa
MAVATMALSSSFAAAAAGSAPWRGVVAAGRAAVGFPPRRRAAALVVRAQAEPEVEPTKEEAATSSSPTPTPSPAAAAPRAKPAASTGLWDVLAFSGPAPERINGRLAMVGFVSALAVEASRGGGLLRPAAGAGSRGSPPPPPCSPRRRWCRCSAARARRRGAAA

SEQ ID NO: 3 - protein, 195 aa, Arabidopsis thaliana
MATASFNMQSVFAGGLTTRKINTNKLFSAGSFPNLKRNYPVGVRCMAEGGPTNEDSSPAPSTSAAQPLPKSPSPPPPMKPKVSTKFSDLLAFSGPAPERINGRLAMVGFVAALAVELSKGENVLAQISDGGVSWFLGTTAILTLASLVPLFKGISVESKSKGIMTSDAELWNGRFAMLGLVALAFTEFVKGGTLV

SEQ ID NO: 4 - protein, 193 aa, Arabidopsis thaliana
MATASFNMQSVFAAPSGVLTTRNIRNTNQLFFKRIAPVGVRCMAQGDPIKEDPSVPSTSTSATPPQMPQSPPPPVSKPKVSTKFGDLLAFSGPAPERINGRLAMVGFVAAIAMELSKGENVFAQISDGGVGWFLGTTALLTLASMVPLFKGIRAEAKSKGFMTSDAELWNGRFAMLGLVALAFTEYVTGGTLV

SEQ ID NO: 5 - protein, 182 aa, Zea mays
MATTVMASATASSPAFSAPGPRQAARLPAGFRGRVAARRAASCCSPGPPSRPMTLPPTRARAAPAARKLGLWDALAFSGPAPERINGRLAMVGFVSALAVEATRGDGILSPGNGAGLAWFAYTAAVLSAASLVPLLQGESAEGRSGRFMTADAELWNGRFAMLGLVALAVTEYLTGAPFVNV

SEQ ID NO: 6 - protein, 169 aa, Zea mays
SAPRRAPARRPASGGVPRPCRRRRAASRCSPGPPSRPMTLPPTRARARRRLLGSWGSGDALAFSGPAPERINGRLAMVGFVSALAVEATRGDGILSQAGNGAGLAWFAYTAAVLSAASLVPLLQGESAEGRSGRFMTADAELWNGRLAMLGLVALAVTEYLTGAPFVNV

SEQ ID NO: 7 - protein, 189 aa, Triticum aestivum
MTTMVALSSFAGAAVVGRPSSWSPAVPHRRTLLVRAQTEPDIESTKDTTSASTSSPTPTPTPSPVAPKPKPKANPSVWDALAFSGPAPERINGRLAMVGFVAALSVEAARGGALLDQAGSGAGLGWFLTTAAVFSVASLVPLLQGQSVESKSSGVWSADAELWNGRFAMLGLVALAVTEFITGTPFVNV

SEQ ID NO: 8 - protein, 193 aa, Triticum aestivum
ARATMVALSSFAGAAVVGRSASWSPAVPRRRALLVRAQTEPDVEPTKETMTSASTSSPSPTPSPSPTPVAPKPKAKANPSVWDALAFSGPAPERINGRLAMVGFVAALSVEAARGGGLLDQVGSGAGLGWFLTTAAVFSVASLVPLLQGQSVESKSSGVWTADAELWNGRFAMLGLVALAVTEFITGTPFVNV

SEQ ID NO: 9 - protein, 206 aa, Triticum aestivum
  MOD_RES (197): variable amino acid
MATMVALSSFAVVGRSATRSPVAAPRRRALVVRAQTEPGMESTKETTSASTSSSSPSTSAAPTPIPAAPKPMTKKANPSVWDALAFSGPAPERINGRLAMVGFVAALTVEAARGGGLLDQAGSGAGLGWFLVTGGVFSVASLVPLLQGQSVESKSSGFWSADAELWNGRFAMLGLVALARTEFITRAPFLTSKTAYXSAAGSFFIS

SEQ ID NO: 10 - protein, 202 aa, Oryza sativa
MAAATMALSSSFAVAAAAAGGAPWRGVVSAGRAAPRRRVALVVRAQSEPEVEPTKEETATSSSSPSPATTPTPSPAAAAPKAKPAASTKLWDVLAFSGPAPERINGRLAMVGFVSALAVEASRGGGLLDQAGSWSGLAWFAATAAVLSAASLVPLLRGETAEARSGGVMSADAELWNGRFAMLGLVALAFTEFLTGSPLVNV

SEQ ID NO: 11 - protein, 211 aa, Zea mays
MAATVMASMSPLASATPGARRAFPVRLLLQASALAPRQRALAVTVRAQSDDAEAEPKEEAAAATTPAPKSKAAATASPGLWDALAFSGPAPERINGRLAMVGFVSALAVEASRGGGLLSQAGSGSGLAWFAATAAVLSVASLVPLLRGDSAEARSGAVMSADAELWNGRSPCSASSRSPSLSTSPAHRSSTRRCVVYFGTVYASQLAAPCA

SEQ ID NO: 12 - DNA, 30 bp, Artificial Sequence
  Description of Artificial Sequence Synthetic primer
cgggatcctttagctttagactagagtccc

SEQ ID NO: 13 - DNA, 38 bp, Artificial Sequence
  Description of Artificial Sequence Synthetic primer
ataagaatgcggccgccatgcagtcagttttcgctgct

SEQ ID NO: 14 - DNA, 30 bp, Artificial Sequence
  Description of Artificial Sequence Synthetic primer
cgggatccattcatgggcaaatcgtattaa

SEQ ID NO: 15 - DNA, 38 bp, Artificial Sequence
  Description of Artificial Sequence Synthetic primer
ataagaatgcggccgcaatggcaacagcatcgttcaac

SEQ ID NO: 16 - DNA, 35 bp, Artificial Sequence
  Description of Artificial Sequence Synthetic primer
ataagaatgaattcatgcagtcagttttcgctgct

SEQ ID NO: 17 - DNA, 36 bp, Artificial Sequence
  Description of Artificial Sequence Synthetic primer
ataagaatgaattcaatggcaacagcatcgttcaac